(19) United States
(12) Patent Application Publication
(10) Pub. No.: US 2018/0085405 A1
(43) Pub. Date: Mar. 29, 2018

(54) TREATMENT OF STROKE BY AMNIOTIC FLUID DERIVED STEM CELL CONDITIONED MEDIA AND PRODUCTS DERIVED THEREOF

(71) Applicant: Creative Medical Technologies, Inc., Phoenix, AZ (US)

(72) Inventors: Thomas Ichim, San Diego, CA (US); Amit Patel, Salt Lake City, UT (US); Santosh Kesari, Santa Monica, CA (US)

(73) Assignee: Creative Medical Technologies, Inc., Phoenix, AZ (US)

(21) Appl. No.: 15/717,810

(22) Filed: Sep. 27, 2017

Related U.S. Application Data
(60) Provisional application No. 62/400,557, filed on Sep. 27, 2016.

(51) Int. Cl. A61K 35/50

(52) U.S. Cl. CPC A61K 35/50

(57) ABSTRACT

Disclosed are compositions of matter useful for treatment of stroke derived from amniotic fluid stem cell produced factors. In one embodiment the invention teaches the use of products derived from amniotic fluid stem cells cultured under basal conditions. In another embodiment the invention teaches the utilization of amniotic stem cell derived products from said amniotic stem cells cultured under conditions of stress. Said amniotic stem cell derived products include small molecules, proteins, peptides, conditioned media, microvesicles, including exosomes and apoptotic bodies. In one embodiment, the invention teaches administration of amniotic fluid stem cells that have been exposed to a stress condition.

### CROSS-REFERENCE TO RELATED APPLICATIONS

This application is related to, and claims the benefit of U.S. Provisional Application No. 62/400,557, filed Sep. 27, 2016. The above-identified priority patent application is incorporated herein by reference in its entirety.

### FIELD OF THE INVENTION

The invention pertains to the field of stroke, more specifically, the invention teaches the use of conditioned media generated from amniotic fluid stem cells for treatment of stroke, more specifically, the invention teaches the generation of therapeutic conditioned media for treatment of stroke subsequent to stressing of said amniotic fluid stem cells.

### BACKGROUND OF THE INVENTION

Stroke is the third leading cause of death and disability in adults in the US. Thrombolytic therapy only benefits about 2% of the ischemic stroke patients. Reduction of tissue plasminogen activator induced hemorrhage and brain injury by free radical spin trapping after embolic focal cerebral ischemia in rats. The dismal record of neurorestorative regimens for stroke both in the laboratory and the clinic solicits an urgent need to develop novel therapies. Because the secondary cellular death that ensues after the initial stroke episode occurs over an extended time. Treatment strategies directed at rescuing these ischemic neurons have the potential to retard the disease progression and even afford restoration of function. The recognition of this delay in secondary stroke-induced pathophysiologic alterations has prompted investigations on neurorestorative treatments, including cell therapy, to salvage the ischemic penumbra and promote functional recovery from stroke. Cell therapy thus offers a new avenue for the treatment and management of stroke.

Unfortunately utilization of stem cells is limited by amount that can be administered intravenously due to pulmonary retention. The invention overcomes this problem by utilizing stem cells from an amply available source: amniotic fluid, and as well utilizing conditioned media, or more particularly, microvesicles derived from conditioned media in order to endow therapeutic properties of stem cells without administration of the stem cells themselves.

### DESCRIPTION OF THE INVENTION

In one embodiment of the invention, disclosed is the unique and unexpected finding that amniotic fluid stem cells may be utilized as a starting material for generation of conditioned media, wherein said conditioned media possesses ability to induce acceleration of post-stroke recovery. Amniotic fluid is routinely collected during amniocentesis procedures. In one embodiment amniotic fluid mononuclear cells are utilized therapeutically in an unpurified manner. In other embodiments amniotic fluid stem cells are substantially purified based on expression of markers such as SSEA-3, SSEA4, Tra-1-60, Tra-1-81 and Tra-2-54, and subsequently administered. In other embodiments cells are cultured, as described in US patent application #20050054093, expanded, and subsequently infused into the patient. Amniotic stem cells are described in the following references [1-3]. One particular aspect of amniotic stem cells that makes them amenable for use in practicing certain aspects of the current invention is their potent growth factor secreting and anti-inflammatory activity [4].

Further embodiments include a method of optimizing therapeutic factor production from said stem or progenitor cells for brain regenerating properties post-stroke through the use of filters that separate compositions based on electrical charge, size or ability to elute from an adsorbent. Numerous techniques are known in the art for purification of therapeutic factors and concentration of said agents. For some particular uses said stem or progenitor cell derived compounds will be sufficient for use as culture supernatants of said cells in media. Currently media useful for this purpose include Roswell Park Memorial Institute (RPMI-1640), Dublecco's Modified Essential Media (DMEM), Eagle's Modified Essential Media (EMEM), Optimem, and Iscove's Media.

Culture conditioned media may be concentrated by filtering/desalting means known in the art including use of Amicon filters with specific molecular weight cut-offs, said cut-offs may select for molecular weights higher than 1 kDa to 50 kDa. Supernatant may alternatively be concentrated using means known in the art such as solid phase extraction using C18 cartridges (Mini-Spe-ed C18-14%, S.P.E. Limited, Concord ON). Said cartridges are prepared by washing with methanol followed by deionized-distilled water. Up to 100 ml of stem cell or progenitor cell supernatant may be passed through each of these specific cartridges before elution, it is understood of one of skill in the art that larger cartridges may be used. After washing the cartridges material adsorbed is eluted with 3 ml methanol, evaporated under a stream of nitrogen, redissolved in a small volume of methanol, and stored at 4.degree. C. Before testing the eluate for activity in vitro, the methanol is evaporated under nitrogen and replaced by culture medium. Said C18 cartridges are used to adsorb small hydrophobic molecules from the stem or progenitor cell culture supernatant, and allows for the elimination of salts and other polar contaminants. It may, however be desired to use other adsorption means in order to purify certain compounds from said stem or progenitor cell supernatant. Said concentrated supernatant may be assessed directly for biological activities useful for the practice of this invention, or may be further purified. Further purification may be performed using, for example, gel filtration using a Bio-Gel P-2 column with a nominal exclusion limit of 1800 Da (Bio-Rad, Richmond Calif.). Said column may be washed and pre-swelled in 20 mM Tris-HCl buffer, pH 7.2 (Sigma) and degassed by gentle swirling under vacuum. Bio-Gel P-2 material be packed into a 1.5.times.54 cm glass column and equilibrated with 3 column volumes of the same buffer. Amniotic fluid stem cell supernatant concentrates extracted by C18 cartridge may be dissolved in 0.5 ml of 20 mM Tris buffer, pH 7.2 and run through the column. Fractions may be collected from the column and analyzed for biological activity. Other purification, fractionation, and identification means are known to one skilled in the art and include anionic exchange chromatography, gas chromatography, high performance liquid chromatography, nuclear magnetic resonance, and mass spectrometry. Administration of supernatant active fractions may be performed locally or systemically.

In order to identify and standardize stem and progenitor cell derived compounds with post-stroke recovery therapeutic properties one embodiment of the invention is the concept of “units of activity” for quantification of said properties. Accordingly, we define 1 Unit as the concentration of said stem or progenitor derived compounds as having sufficient activity to stimulate a biological response in an in vitro setting to a certain degree. Depending on use, this can be stimulation of a standardized cell culture to proliferate by a certain percentage, in other desired uses the Unit may designate the amount needed to inhibit differentiation a specified culture condition by a defined percentage. In a specific embodiment, one Unit is the activity sufficient to inhibit production of the inflammatory compound TNF-alpha by 50% in a culture of 0.5 ug/ml endotoxin stimulated culture of RAW macrophage cell line cultured at a concentration of 10(4) cells per plate in flat-bottom 96 well plates. Other methods of quantifying activity may be chosen based on other desired biological activities relevant to the pathology of post-stroke inflammation. Without being bound to mechanism, said activities include: inhibition of inflammation; inhibition of brain fibrosis; stimulation of endogenous neural stem cells.

As used herein the phrase “culture medium” refers to a solid or a liquid substance used to support the growth of stem cells and maintain them in an undifferentiated state. Preferably, the phrase “culture medium” as used herein refers to a liquid substance capable of maintaining the stem cells in an undifferentiated state. The culture medium used by the present invention can be a water-based medium which includes a combination of substances such as salts, nutrients, minerals, vitamins, amino acids, nucleic acids, proteins such as cytokines, growth factors and hormones, all of which are needed for cell proliferation and are capable of maintaining the stem cells in an undifferentiated state. For example, a culture medium according to this aspect of the present invention can be a synthetic tissue culture medium such as Ko-DMEM (Gibco-Invitrogen Corporation products, Grand Island, N.Y., USA), DMEM/F12 (Biological Industries, Biet Haemek, Israel), Mab ADCB medium (HyClone, Utah, USA) or DMEM/F12 (Biological Industries, Biet Haemek, Israel) supplemented with the necessary additives as is further described hereinunder. Preferably, all ingredients included in the culture medium of the present invention are substantially pure, with a tissue culture grade.

In another embodiment of the invention, disclosed is the finding that surprisingly identified microparticles isolated from amniotic fluid stem cells. Said microparticles retain some of the functions of the amniotic fluid stem cells from which they are derived and are typically therapeutically useful for the same treatments as the amniotic fluid stem cells. The microparticles are advantageous over the corresponding stem cells because they are smaller and less complex, thereby being easier to produce, maintain, store and transport, and have the potential to avoid some of the regulatory issues that surround stem cells. Additionally, said microparties do not possess the limitation of pulmonary retention that is found when cells are administered intravenously. The microparticles can be produced continuously, by isolation from conditioned media, for example in a bioreactor such as a multi-compartment bioreactor, which allows for large scale production and the provision of an “off-the-shelf” therapy. The multi-compartment bioreactor is typically a two-compartment bioreactor. The invention teaches that culture of amniotic fluid stem cells in a multi-compartment bioreactor, results in partial differentiation of the stem cells, into stem cells in a more differentiated form. This differentiation in culture does not require the addition of an agent to induce differentiation. This differentiation typically requires a culture period of at least one week, at least two weeks or at least three weeks. The changes to the stem cells that occur in culture in a multi-compartment bioreactor are reflected by the microparticles produced by the cultured stem cells. Therefore, by culturing stem cells in a multi-compartment bioreactor, it is possible to induce differentiation of the cells. Accordingly, microparticles from partially differentiated stem cells can be produced by harvesting microparticles from stem cells cultured in a multi-compartment bioreactor, typically for at least one week, at least two weeks, at least three weeks, at least four weeks, at least five weeks or at least six weeks. Optionally, the stem cells have been cultured for no more than ten weeks. In one embodiment, the invention provides a method of producing microparticles by isolating the microparticles from partially-differentiated amniotic fluid stem cells.

Culture of amniotic fluid stem cells may be conducted under conditions of stress in order to augment production of microparticles with therapeutic benefit in stroke, or stroke rehabilitation. One example of stress administered to said amniotic fluid stem cells is treatment with TNF-alpha. Concentrations of TNF-alpha range from 1 pg/ml to 100 ng/ml, more preferably approximately 10 ng/ml. Culture conditions are well known in the art for exposing stem cells to inflammatory cytokines and include culture for 1 hour to 72 hours, more preferably approximately 24 hours. For the practice of the invention, the term “microparticle” is an extracellular vesicle of 30 to 1000 nm diameter that is released from a cell. It is limited by a lipid bilayer that encloses biological molecules. The term “microparticle” is known in the art and encompasses a number of different species of microparticle, including a membrane particle, membrane vesicle, microvesicle, exosome-like vesicle, exosome, ectosome-like vesicle, ectosome or exovesicle. The different types of microparticle are distinguished based on diameter, subcellular origin, their density in sucrose, shape, sedimentation rate, lipid composition, protein markers and mode of secretion (i.e. following a signal (inducible) or spontaneously (constitutive)). Microparticles are thought to play a role in intercellular communication by acting as vehicles between a donor and recipient cell through direct and indirect mechanisms. Direct mechanisms include the uptake of the microparticle and its donor cell-derived components (such as proteins, lipids or nucleic acids) by the recipient cell, the components having a biological activity in the recipient cell. Indirect mechanisms include microvesicle-recipient cell surface interaction, and causing modulation of intracellular signalling of the recipient cell. Hence, microparticles may mediate the acquisition of one or more donor cell-derived properties by the recipient cell. It has been observed that, despite the efficacy of stem cell therapies in animal models, the stem cells do not appear to engraft into the host. Accordingly, the mechanism by which stem cell therapies are effective is not clear. Without wishing to be bound by theory, the inventors believe that the microparticles secreted by amniotic fluid stem cells play a role in the therapeutic utility of these cells and are therefore therapeutically useful themselves.

For the practice of the invention, microparticles, or microvesicles are isolated from tissue culture. The term “isolated” for the purpose of the practice of the invention indicates that the microparticle, microparticle population, cell or cell population to which it refers is not within its natural environment. The microparticle, microparticle population, cell or cell population has been substantially separated from surrounding tissue. In some embodiments, the microparticle, microparticle population, cell or cell population is substantially separated from surrounding tissue if the sample contains at least about 75%, in some embodiments at least about 85%, in some embodiments at least about 90%, and in some embodiments at least about 95% microparticles and/or stem cells. More specifically, the sample is substantially separated from the surrounding tissue if the sample contains less than about 25%, in some embodiments less than about 15%, and in some embodiments less than about 5% of materials other than the microparticles and/or stem cells. Such percentage values refer to percentage by weight. The term encompasses cells or microparticles which have been removed from the organism from which they originated, and exist in culture. The term also encompasses cells or microparticles which have been removed from the organism from which they originated, and subsequently re-inserted into an organism. The organism which contains the re-inserted cells may be the same organism from which the cells were removed, or it may be a different organism.

In one embodiment, the invention provides amniotic fluid stem cell derived microparticle. The microparticle may be an exosome, microvesicle, membrane particle, membrane vesicle, exosome-like vesicle, ectosome-like vesicle, ectosome or exovesicle. Typically, the microparticle is an exosome. The microparticle may be derived from an amniotic fluid stem cell that has been cultured in an environment that allows stem cell differentiation into the neural lineage. In another embodiment of the invention said amniotic fluid stem cells are exposed to conditions of stress. The microparticle may be isolated from partially-differentiated or differentiated amniotic fluid stem cells. In one embodiment, an environment that allows stem cell differentiation is a multi-compartment bioreactor, typically where the cells are cultured for more than seven days. The microparticle may be derived from an amniotic fluid stem cell line. In some embodiments, the microparticle is derived from a stem cell line that does not require serum to be maintained in culture. The microparticle may have a size of between 30 nm and 1000 nm, or between 30 and 200 nm, or between 30 and 100 nm, as determined by electron microscopy; and/or a density in sucrose of 1.1-1.2 g/ml. The microparticle may comprise RNA, or may contain RNA. The RNA may be mRNA, miRNA, and/or any other small RNA. The microparticle may comprise one, two, three or four of hsa-miR-1246, hsa-miR-4492, hsa-miR-4488 and hsa-miR-4532. The microparticle may comprise one or more lipids, typically selected from ceramide, cholesterol, sphingomyelin, phosphatidylserine, phosphatidylinositol, phosphatidylcholine. The microparticle may comprise one or more tetraspanins, typically CD63, CD81, CD9, CD53, CD82 and/or CD37. The microparticle may comprise one or more of TSG101, Alix, CD109, thy-1 and CD133. The microparticle may comprise at least one biological activity of an amniotic fluid stem cell or a neural stem cell-conditioned medium. At least one biological activity may be a tissue regenerative activity. The microparticle of the invention is typically isolated or purified.

Cells for use in the invention are derived from amniotic fluid for treatment of ischemic neurological conditions. The cells described in the invention are immortal in culture, maintain euploidy for >1 year in culture, share markers with human ES cells, and are capable of differentiating into all three germ layers of the developing embryo, Endoderm, Mesoderm and Ectoderm. In a preferred embodiment the regenerative amniotic fluid cells are found in the amnion harvested during the second trimester of human pregnancies. It is known that amniotic fluid contains multiple morphologically-distinguishable cell types, the majority of the cells are prone to senescence and are lost from cultures. In one embodiment, fibronectin coated plates and culture conditions described in U.S. Pat. No. 7,569,385 are used to grow cells from amniotic fluid harvests from normal 16-18 week pregnancies. The cells of the invention are of fetal origin, and have a normal diploid karyotype. Growth of the amniotic fluid stem cells as described in the invention for use in neurological ischemic conditions results in cells that are multipotent, as several main cell types have been derived from them. As used herein, the term “multipotent” refers to the ability of amniotic fluid regenerative cells to differentiate into several main cell types. The MAFSC cells may also be propagated under specific conditions to become “pluripotent.” The term “pluripotent stem cells” describes stem cells that are capable of differentiating into any type of body cell, when cultured under conditions that give rise to the particular cell type. The Amniotic fluid regenerative cells are preferably isolated from humans. However, the Amniotic fluid regenerative cells may be isolated in a similar manner from other species. Examples of species that may be used to derive the Amniotic fluid regenerative cells include but are not limited to mammals, humans, primates, dogs, cats, goats, elephants, endangered species, cattle, horses, pigs, mice, rabbits, and the like.

The amniotic fluid-derived cells and MAFSC can be recognized by their specific cell surface proteins or by the presence of specific cellular proteins. Typically, specific cell types have specific cell surface proteins. These surface proteins can be used as “markers” to determine or confirm specific cell types. Typically, these surface markers can be visualized using antibody-based technology or other detection methods. One method of characterizing cellular markers, FACS analysis, is described in Example 3.

The surface markers of the isolated MAFSC cells derived from independently-harvested amniotic fluid samples were tested for a range of cell surface and other markers, using monoclonal antibodies and FACS analysis (see Example 3 and Table 1). These cells can be characterized by the following cell surface markers: SSEA3, SSEA4, Tra-1-60, Tra-1-81, Tra-2-54, as shown in FIG. 3. The MAFSC cells can be distinguished from mouse ES cells in that the MAFSC cells do not express the cell surface marker SSEA1. Additionally, MAFSC express the stem cell transcription factor Oct-4. The MAFSC cells can be recognized by the presence of at least one, or at least two, or at least three, or at least four, or at least five, or at least six, or all of the following cellular markers SSEA3, SSEA4, Tra-1-60, Tra-1-81, Tra-2-54 and Oct-4.

In some embodiments of the present invention, the SSEA3 marker is expressed in a range of from about 90%, 92%, 94% to about 96%, 98%, 99%, or 100% of the cells in the MAFSC culture. The SSEA4 marker can be expressed, for example, in a range of from about 90%, 92%, 94% to about 96%, 98%, 99%, or 100% of the cells in the MAFSC culture. In some embodiments of the present invention, the Tra-1-60 marker expressed, for example, in a range of from about 60%, 65%, or 70% to about 85%, 90%, or 95% of the cells in the MAFSC culture. In some embodiments of the present invention, the Tra-1-81 marker is expressed in a range of from about 70%, 75%, or 80% to about 85%, 90%, or 95% of the cells in the MAFSC culture. The Tra-2-84 marker can be expressed, for example, in a range of from about 55%, 60%, 65%, or 70% to about 80%, 90%, or 95% of the cells in the MAFSC culture. In some embodiments of the present invention, the Oct-4 marker is expressed in a range of from about 25%, 30%, 35%, or 40% to about 45%, 55%, 65%, or 70% of the cells in the MAFSC culture.

The MAFSC cultures express very little or no SSEA-1 marker. In addition to the embryo stem cell markers SSEA3, SSEA4, Tra1-60, Tra1-81, Tra2-54, Oct-4 the amniotic fluid regenerative cells also expressed high levels of the cell surface antigens that are normally found on human mesenchymal stem cells, but not normally on human embryo stem cells (M F Pittinger et al., Science 284:143-147, 1999; S Gronthos et al., J. Cell Physiol. 189:54-63, 2001). This set of markers includes CD13 (99.6%) aminopeptidase N, CD44 (99.7%) hyaluronic acid-binding receptor, CD49b (99.8%) collagen/laminin-binding integrin alpha2, and CD105 (97%) endoglin. The presence of both the embryonic stem cell markers and the hMSC markers on the MAFSC cell cultures indicates that amniotic fluid-derived MAFSC cells, grown and propagated as described here, represent a novel class of human stem cells that combined the characteristics of hES cells and of hMSC cells.

In some embodiments of the invention, at least about 90%, 94%, 97%, 99%, or 100% of the cells in the culture express CD13. In additional embodiments, at least about 90%, 94%, 97%, 99%, or 100% of the cells in the culture express CD44. In some embodiments of the invention, a range from at least about 90%, 94%, 97%, 99%, 99.5%, or 100% of the cells in the culture express CD49b. In further embodiments of the invention, a range from at least about 90%, 94%, 97%, 99%, 99.5%, or 100% of the cells in the culture express CD105.

Microparticles for the practice of the invention are typically isolated from stem cell conditioned media. The “conditioned medium” (CM) may be a growth medium for stem cells, which has been used to culture a mass culture of stem cells for at least about 12 hours, at least about 24 hours, at least about 48 hours or least about 72 hours, typically up to 168 hours (7 days), removed and sterilized by any suitable means, preferably by filtration, prior to use, if required. The media in which cells are cultured contains various growth factors. In some cases serum free media is used so as to avoid contamination with fetal calf serum or other serum associated exosomes. Alternatively, microparticles may be harvested from a two-compartment bioreactor which allows the cell culture, and hence the conditioned media, to be maintained for longer periods of time, for example at least 2 weeks, at least 3 weeks, at least 4 weeks, at least 5 weeks, at least 6 weeks or more. The system maintains the cells and secreted microparticles within a small cell compartment (approximately 15 ml) which is separated from a larger reservoir of medium by a 10 kDa semi-permeable membrane. This allows the efficient removal of metabolic waste products while effectively maintaining an extremely high cell density to maximize microparticle production. The microparticles may be separated from other media components based on molecular weight, size, shape, hydrodynamic radius, composition, charge, substrate-ligand interaction, absorbance or scattering of electromagnetic waves, or biological activity. In one embodiment, the conditioned media is filtered using a filter of appropriate size to separate the desired microparticle, for example a 100K MWCO filter. Optionally, the stem cell-conditioned medium is concentrated prior to the isolation of the microparticles by subjecting the concentrated NSC-conditioned medium to size exclusion chromatography. The UV absorbant fractions can then be selected for isolation of the microparticles of interest.

For the practice of the invention different microparticles can be isolated from the media by using different isolation techniques and parameters. For example, exosomes have a vesicle density of 1.13-1.19 g/mL and can be isolated by differential centrifugation and sucrose gradient ultracentrifugation at 100,000-200,000 g. Microvesicles can be isolated by filtration (100K MWCO) and differential centrifugation at 18,000-20,000 g. Membrane particles have a density of 1.04-01.07 g/ml and Exosome-like vesicles have a density of 1.1 g/ml. A typical production method comprises: culturing stem cells to produce conditioned media; removing cell debris by centrifugation at 1500 rpm; isolating microvesicles (<1000 kDa) by ultrafiltration through a 100K MWCO filter or isolating exosomes (30-100 nm) by ultracentrifugation at 120,000 g; followed by quantification using a BCA protein assay. Production of stem cell derived particles is described in the following references and incorporated herein to allow for practice of the invention.

Ma et al. [5], generated mesenchymal stem cell exosomes to treat an acute myocardial infarction model that was created by ligation of the left anterior decedent coronary artery (LAD) in rats. Various source exosomes (400 μg of protein) were infused via the tail vein immediately after LAD ligation. The cardiac function was evaluated by using echocardiography after different treatments for 1 and 5 weeks, respectively. Endothelial cell proliferation, migration, and tube-like structure formation, as well as chick allantoic membrane assay, were used to evaluate the angiogenetic effects of Akt-Exo. The results indicated that cardiac function was significantly improved in the animals treated with Akt-Exo. In addition, Akt-Exo significantly accelerated endothelial cell proliferation and migration, tube-like structure formation in vitro, and blood vessel formation in vivo. The expression of platelet-derived growth factor D (PDGF-D) was significantly upregulated in Akt-Exo. However, the angiogenesis was abrogated in endothelial cells treated with the exosomes obtained from MSCs transfected with PDGF-D-siRNA.

Hu et al. [6], assessed the effects of ASCs-derived exosomes (ASCs-Exos) in cutaneous wound healing. They found that ASCs-Exos could be taken up and internalized by fibroblasts to stimulate cell migration, proliferation and collagen synthesis in a dose-dependent manner, with increased genes expression of N-cadherin, cyclin-1, PCNA and collagen I, III. In vivo tracing experiments demonstrated that ASCs-Exos can be recruited to soft tissue wound area in a mouse skin incision model and significantly accelerated cutaneous wound healing. Histological analysis showed increased collagen I and III production by systemic administration of exosomes in the early stage of wound healing, while in the late stage, exosomes might inhibit collagen expression to reduce scar formation. Collectively, their findings indicate that ASCs-Exos can facilitate cutaneous wound healing via optimizing the characteristics of fibroblasts.

Nong et al. [7], used human inducible IPC cells (hiPSCs) that were efficiently induced into hiPSC-MSCs with typical MSC characteristics. hiPSC-MSCs-Exo had diameters ranging from 50 to 60 nm and expressed exosomal markers (CD9, CD63 and CD81). Hepatocyte necrosis and sinusoidal congestion were markedly suppressed with a lower Suzuki score after hiPSC-MSCs-Exo administration. The levels of the hepatocyte injury markers AST and ALT were significantly lower in the treated group than in the control group. Inflammatory markers, such as tumor necrosis factor (TNF)-α, interleukin (IL)-6 and high mobility group box 1 (HMGB1), were significantly reduced after administration of hiPSC-MSCs-Exo, which suggests that the exosomes have a role in suppressing the inflammatory response. Additionally, in liver tissues from the experimental group, the levels of apoptotic markers, such as caspase-3 and bax, were significantly lower and the levels of oxidative markers, such as glutathione (GSH), glutathione peroxidase (GSH-Px) and superoxide dismutase (SOD), were significantly higher than in the control group.

In one embodiment of the invention amniotic fluid stem cells are cultured in a manner to allow viability. Numerous culture techniques are known in the art. Tissue culture solutions (media) that allow for stem cell viability include Roswell Park Memorial Institute (RPMI-1640), Dublecco's Modified Essential Media (DMEM), Eagle's Modified Essential Media (EMEM), Optimem, and Iscove's Media. Said media is usually supplemented with a source of serum, or alternatively serum-free media may be used. Serum from fetal calves is typically used at a concentration ranging from 2%-20%, more preferably at approximately 10%. In some embodiments, said fetal calf serum is heat-inactivated by incubation at 55 Celsius for 1 hour in order to neutralize complement activity. In other embodiments human serum is used as a substitute for fetal calf serum. Conditioned media is collected from cells that are originally plated at a concentration between 20-8000 cells/cm(2), more preferably between 2000-8000 cells/cm(2), and more preferably at an approximate concentration of 4000 cells/cm(2). One of skill in the art may with minimal experimentation identify ideal concentration of cells to be cultured based on assessment of viability, growth factor production, and generation of anti-inflammatory. Conditioned media may be collected at 24-72 hours of culture, filtered to remove cellular debris and depending on the concentration of brain regenerative compounds desired, may be concentrated. Means of concentration are known to one of skill in the art. For example molecular weight filter such as an Amicon 3000 Stir Cell can be used to reduce the volume and at the same time remove low molecular weight salts. Alternatively concentration of brain regenerative components of the conditioned media may be achieved by means of column chromatography; or, lyophilization to remove the water in the medium, effectively concentrating the effective components. Concentrated conditioned media can subsequently be re-mixed with a suitable solution and administered intravenously to induce acceleration of post-stroke recovery.

In certain embodiments amniotic fluid stem cells may be “activated” ex vivo by a brief culture in hypoxic conditions in order to upregulate production of therapeutic factors. Without being bound to theory, said factors may be upregulated by nuclear translocation of the HIF-1 transcription factor. Hypoxia may be achieved by culture of cells in conditions of 0.1% oxygen to 10% oxygen, preferably between 0.5% oxygen and 5% oxygen, and more preferably around 1% oxygen. Cells may be cultured for a variety of timepoints ranging from 1 hour to 72 hours, more preferably from 13 hours to 59 hours and more preferably around 48 hours. In addition to induction of hypoxia, other therapeutic properties can be endowed unto said stem or progenitor cells through treatment ex vivo with factors such as de-differentiating compounds, proliferation inducing compounds, or compounds known to endow and/or enhance stem or progenitor cells to possess properties useful for the practice of the current invention. In one embodiment cells are cultured with an inhibitor of the enzyme GSK-3 in order to enhance expansion of cells with pluripotent characteristics while not increasing the rate of differentiation, or inducing neural differentiation [8, 9]. Examples of GSK-3 inhibitors include lithium salts [10-13]. In another embodiment, cells are cultured in the presence of a DNA methyltransferase inhibitor such as 5-azacytidine in order to endow a “de-differentiation” effect.

Various aspects of the invention relating to the above are enumerated in the following paragraphs:

Aspect 1: A method of accelerating post-stroke recovery comprising administering a conditioned media-derived product generated from an amniotic fluid stem cell.

Aspect 2: The method of aspect 1, wherein said amniotic fluid stem cell comprises a mixture of amniotic fluid derived stem or progenitor cells.

Aspect 3: The method of aspect 1, wherein said conditioned media is a supernatant of a cultured cell population.

Aspect 4: The method of aspect 3, wherein said supernatant is obtained by culturing viable stem or progenitor cells under conditions that are physiological or near-physiological.

Aspect 5: The method of aspect 4, wherein said supernatant is obtained by culturing viable stem or progenitor cells under conditions that are non-physiological.

Aspect 6: The method of aspect 2, wherein said supernatant of a cultured cell population is substantially free of cellular debris.

Aspect 7: The method of aspect 2, wherein said cultured cells are exposed to conditions selected from a group comprising of: a) exposure to hypoxia; b) treatment with a histone deacetylase inhibitor; c) treatment with a growth factor; d) treatment with a DNA methyltransferase inhibitor; and e) exposure to hyperthermia.

Aspect 8: The method of aspect 7, wherein said growth factor is selected from a group comprising of: a WNT signaling agonist, TGF-b, bFGF, IL-6, SCF, BMP-2, thrombopoietin, EPO, IGF-1, IL-11, IL-5, Flt-3/Flk-2 ligand, fibronectin, LIF, HGF, NFG, angiopoietin-like 2 and 3, G-CSF, GM-CSF, Tpo, Shh, Wnt-3a, Kirre, or a mixture thereof.

Aspect 9: The method of aspect 5, wherein said non-physiological condition is a stress condition.

Aspect 10: The method of aspect 9, wherein said stress condition is an inflammatory condition.

Aspect 11: The method of aspect 9, wherein said inflammatory condition is exposure to an inflammatory cytokine.

Aspect 12: The method of aspect 11, wherein said inflammatory cytokine is IL-1.

Aspect 13: The method of aspect 11, wherein said inflammatory cytokine is IL-6.

Aspect 14: The method of aspect 11, wherein said inflammatory cytokine is TNF-alpha.

Aspect 15: The method of aspect 11, wherein said inflammatory cytokine is IL-2.

Aspect 16: The method of aspect 11, wherein said inflammatory cytokine is IL-8.

Aspect 17: The method of aspect 11, wherein said inflammatory cytokine is IL-12.

Aspect 18: The method of aspect 11, wherein said inflammatory cytokine is IL-11.

Aspect 19: The method of aspect 11, wherein said inflammatory cytokine is IL-15.

Aspect 20: The method of aspect 11, wherein said inflammatory cytokine is IL-17.

Aspect 21: The method of aspect 11, wherein said inflammatory cytokine is IL-33.

Aspect 22: The method of aspect 1, wherein said amniotic fluid stem cell is obtained from amniocentesis.

Aspect 23: The method of aspect 1, wherein said amniotic fluid stem cell is obtained from amniotic membranes.

Aspect 24: The method of aspect 1, wherein said amniotic fluid stem cell is of mesenchymal origin.

Aspect 25: The method of aspect 1, wherein said stroke is an ischemic stroke.

Aspect 26: The method of aspect 1, wherein said stroke is a hemorrhagic stroke.

Aspect 27. The method of aspect 1, wherein said stroke is a transient ischemic attack.

Aspect 28: The method of aspect 1, wherein said stroke is a traumatic brain injury.

Aspect 29. The method of aspect 28, wherein said traumatic brain injury is chronic traumatic encephalopathy.

Aspect 30: The method of aspect 28, wherein said stroke is caused by vascular disease of the brain.

Aspect 31; The method of aspect 1, wherein said amniotic cells with regenerative activity are amniotic fluid stem cells.

Aspect 32: The method of aspect 1, wherein said amniotic cells with regenerative activity are amniotic fluid mesenchymal stem cells.

Aspect 33: The method of aspect 1, wherein said amniotic cells with regenerative activity possess an epitheliod morphology.

Aspect 34: The method of aspect 1, wherein said amniotic cells with regenerative activity possess expression of the markers SSEA3, SSEA4, Tra1-60, Tra1-81, Tra2-54, Oct-4 and CD105.

Aspect 35: The method of aspect 35, wherein said amniotic fluid cells are capable of differentiating into bone, cartilage and adipose tissue.

Aspect 36: The method of aspect 35, wherein said amniotic fluid cells possess less than 5% expression of SSEA1.

Aspect 37: The method of aspect 35, wherein said amniotic fluid cells are characterized by senescence after about 60 population doubling.

Aspect 38: The method of aspect 35, wherein said amniotic fluid cells are characterized by senescence after about 300 population doubling.

Aspect 39: The method of aspect 35, wherein said amniotic fluid cells are derived from a mammal.

Aspect 40: The method of aspect 35, wherein said mammal possesses a hemochorial placenta.

Aspect 41: The method of aspect 35, wherein said mammal is a human.

Aspect 42: The method of aspect 1, wherein said amniotic fluid is extracted in the first trimester.

Aspect 43: The method of aspect 1, wherein said amniotic fluid is extracted in the second trimester.

Aspect 44: The method of aspect 1, wherein said amniotic fluid is extracted in the third trimester.

Aspect 45: The method of aspect 1, wherein said amniotic fluid cells express one or more markers selected from a group comprising of: HLA class I, CD13, CD44, and CD49b.

Aspect 46: The method of aspect 1, wherein said amniotic fluid cells with regenerative activity are generated by the steps comprising of: a) harvesting amniotic fluid; b) centrifuging the amniotic fluid; c) plating cells onto plates coated with fibronectin in medium with 2% serum; d) selecting colonies which adhere to the plates; and e) isolating mortal, epithelioid morphology cells.

Aspect 47: The method of aspect 1, wherein said amniotic fluid derived cells with regenerative potential possess ability to inhibit secretion of inflammatory cytokines.

### REFERENCES

1. Bossolasco, P., et al., Molecular and phenotypic characterization of human amniotic fluid cells and their differentiation potential. Cell Res, 2006. 16(4): p. 329-36.
2. Sartore, S., et al., Amniotic mesenchymal cells autotransplanted in a porcine model of cardiac ischemia do not differentiate to cardiogenic phenotypes. Eur J Cardiothorac Surg, 2005. 28(5): p. 677-84.
3. Prusa, A. R., et al., Neurogenic cells in human amniotic fluid. Am J Obstet Gynecol, 2004. 191(1): p. 309-14.
4. Tsai, M. S., et al., Clonal amniotic fluid-derived stem cells express characteristics of both mesenchymal and neural stem cells. Biol Reprod, 2006. 74(3): p. 545-51.
5. Ma, J., et al., Exosomes Derived From Akt-Modified Human Umbilical Cord Mesenchymal Stem Cells Improve Cardiac Regeneration and Promote Angiogenesis via Activating Platelet-Derived Growth Factor D. Stem Cells Transl Med, 2016.
6. Hu, L., et al., Exosomes derived from human adipose mensenchymal stem cells accelerates cutaneous wound healing via optimizing the characteristics of fibroblasts. Sci Rep, 2016. 6: p. 32993.
7. Nong, K., et al., Hepatoprotective effect of exosomes from human-induced pluripotent stem cell-derived mesenchymal stromal cells against hepatic ischemia-reperfusion injury in rats. Cytotherapy, 2016.
8. Gao, L., et al., Glycogen synthase kinase 3 (GSK3)-inhibitor SB216763 promotes the conversion of human umbilical cord mesenchymal stem cells into neural precursors in adherent culture. Hum Cell, 2016.
9. Chuang, D. M., Z. Wang, and C. T. Chiu, GSK-3 as a Target for Lithium-Induced Neuroprotection Against Excitotoxicity in Neuronal Cultures and Animal Models of Ischemic Stroke. Front Mol Neurosci, 2011. 4: p. 15.
10. Ferensztajn-Rochowiak, E. and J. K. Rybakowski, The effect of lithium on hematopoietic, mesenchymal and neural stem cells. Pharmacol Rep, 2016. 68(2): p. 224-30.
11. Dell'Osso, L., et al., A new look at an old drug: neuroprotective effects and therapeutic potentials of lithium salts. Neuropsychiatr Dis Treat, 2016. 12: p. 1687-703.
12. Yu, Z., et al., Lithium chloride attenuates the abnormal osteogenic/adipogenic differentiation of bone marrow-derived mesenchymal stem cells obtained from rats with steroid-related osteonecrosis by activating the beta-catenin pathway. Int J Mol Med, 2015. 36(5): p. 1264-72.
13. Zhu, Z., et al., Lithium stimulates human bone marrow derived mesenchymal stem cell proliferation through GSK-3beta-dependent beta-catenin/Wnt pathway activation. FEBS J, 2014. 281(23): p. 5371-89.

1. A method of accelerating post-stroke recovery comprising administering a conditioned media-derived product generated from an amniotic fluid stem cell.

2. The method of claim 1, wherein said amniotic fluid stem cell comprises a mixture of amniotic fluid derived stem or progenitor cells.

3. The method of claim 1, wherein said conditioned media is a supernatant of a cultured cell population.

4. The method of claim 3, wherein said supernatant is obtained by culturing viable stem or progenitor cells under conditions that are physiological or near-physiological.

5. The method of claim 4, wherein said supernatant is obtained by culturing viable stem or progenitor cells under conditions that are non-physiological.

6. The method of claim 2, wherein said supernatant of a cultured cell population is substantially free of cellular debris.

7. The method of claim 2, wherein said cultured cells are exposed to conditions selected from a group comprising of: a) exposure to hypoxia; b) treatment with a histone deacetylase inhibitor; c) treatment with a growth factor; d) treatment with a DNA methyltransferase inhibitor; and e) exposure to hyperthermia.

8. The method of claim 7, wherein said growth factor is selected from a group comprising of: a WNT signaling agonist, TGF-b, bFGF, IL-6, SCF, BMP-2, thrombopoietin, EPO, IGF-1, IL-11, IL-5, Flt-3/Flk-2 ligand, fibronectin, LIF, HGF, NFG, angiopoietin-like 2 and 3, G-CSF, GM-CSF, Tpo, Shh, Wnt-3a, Kirre, or a mixture thereof.

9. The method of claim 5, wherein said non-physiological condition is a stress condition.

10. The method of claim 9, wherein said stress condition is an inflammatory condition.

11. The method of claim 9, wherein said inflammatory condition is exposure to an inflammatory cytokine.

12. The method of claim 11, wherein said inflammatory cytokine is IL-1.

13. The method of claim 11, wherein said inflammatory cytokine is IL-6.

14. The method of claim 11, wherein said inflammatory cytokine is TNF-alpha.

15. The method of claim 11, wherein said inflammatory cytokine is IL-2.

16. The method of claim 11, wherein said inflammatory cytokine is IL-8.

17. The method of claim 11, wherein said inflammatory cytokine is IL-12.

18. The method of claim 11, wherein said inflammatory cytokine is IL-11.

19. The method of claim 11, wherein said inflammatory cytokine is IL-15.

20. The method of claim 11, wherein said inflammatory cytokine is IL-17.
